(19) United States
(12) Patent Application Publication
(10) Pub. No.: US 2007/0065847 A1
(43) Pub. Date: Mar. 22, 2007

(54) Degeneratively Labeled Probes

(75) Inventors: Eugeni A. Namsaraev, Sunnyvale, CA (US); Nicholas Naclerio, Menlo Park, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, INC., Santa Clara, CA (US)

(21) Appl. No.: 11/464,174

(22) Filed: Aug. 11, 2006

Related U.S. Application Data
(60) Provisional application No. 60/707,236, filed on Aug. 11, 2005.

(51) Int. Cl. C12Q 1/68; C12P 19/34

(52) U.S. Cl. 435/6; 435/91.2

(57) ABSTRACT

The invention is directed to methods for determining whether any of group of analytes of interest are present in a sample, and optionally, in at least one subsequent determination, which of the analytes are present. The invention is also directed towards methods for the rapid and efficient determination of the presence or absence of the members of groups of analytes. The invention also contemplates array devices useful for practicing the aforementioned methods, particularly in a screening stage, which comprise fewer array elements than are required to uniquely identify all the analytes.

### RELATED APPLICATIONS

The present application claims priority to U.S. Provisional Application Nos. 60/707,236 filed Aug. 11, 2005 the disclosure of which is incorporated herein by reference in its entirety.

### FIELD OF THE INVENTION

The invention relates generally to methods for detecting the presence of one or more polynucleotide sequences in a sample, and more particularly, to the use of degenerately labeled probes to make such determinations.

### BACKGROUND

DNA-based identification is a key technology in many fields, including medicine, forensics, food production, animal husbandry, and the like, e.g. Jobling et al., Nature Reviews Genetics, 5: 739-751 (2004); Jo et al., Semin. Oncol., 32: 11-23 (2005); Woo et al., J. Clin. Microbiol., 41: 1996-2001 (2003). In particular, DNA amplification technologies, such as polymerase chain reactions (PCRs) and nucleic acid sequence-based amplifications (NASBAs), have found applications in all of these areas, including applications for viral and bacterial detection, viral load monitoring, detection of rare and/or difficult-to-culture pathogens, rapid detection of bio-terror threats, detection of minimal residual disease in cancer patients, food pathogen testing, blood supply screening, and the like, e.g. Mackay, Clin. Microbiol. Infect., 10: 190-212 (2004); Bernard et al, Clinical Chemistry, 48: 1178-1185 (2002). Key reasons for such widespread use are speed and ease of use (typically performed within a few hours using standardized kits and relatively simple and low cost instruments), sensitivity (often a few tens of copies of a target sequence in a sample can be detected), and robustness (poor quality samples or preserved samples, such as forensic samples or fixed tissue samples are readily analyzed), Strachan and Read, Human Molecular Genetics 2 (John Wiley & Sons, New York, 1999).

Despite the advances in nucleic acid amplification techniques that are reflected in such widespread applications, there has been limited achievement in performing these techniques in parallel within the same sample, i.e. in multiplexed assays, where multiple target sequences are simultaneously amplified and detected in the same reaction mixture, e.g. Einifro et al, Clin. Microbiol. Rev., 13: 559-570 (2000); Henegarin et al, Biotechniques, 23: 504-511 (1997).

Microarray technology has provided an alternative approach for making simultaneous measurements on samples containing multiple polynucleotide analytes, e.g. U.S. Pat. Nos. 5,324,633 and 5,445,934, Lockhart et al., Nat. Biotechnol., 14: 1675-80 (1996) and Wodicka et al., Nat. Biotechnol., 15:1359-67 (1996). This technology follows the dominant approach in multiplexed analysis which is to obtain data on every analyte of interest present in the sample. As such, multiplexed assays are primarily concerned with resolving and distinguishing among the plurality of analytes targeted. The number of targets is generally limited in some manner by either the recognition event (e.g. specificity of a probe for a target; the difficulty in designing primer sets that do not interact to negatively effect the amplification) or the detection method (e.g. broad absorption or emission profiles of a chromophore or lumophore limit the number of such labels that can be independently determined). Nonetheless, much work in the field is directed to improving the multiplex capabilities of existing methods by being able to detect or resolve more analytes within a single process.

There is a recognition that some situations require the ability to identify whether one or any member of a large set of possibilities is present without necessarily determining which member. Although some technologies, such as the microarray, can provide such information (and more), a more flexible and cost effective manner of obtaining no more than the necessary information would be of great significance. Moreover, when, for example, there is a terrorist release of pathogens, an outbreak of highly virulent bacteria or viruses, or in certain acute medical conditions, the ability to rapidly alter the set of targets being analyzed is highly desirable. Thus methods that can both screen for and/or specify the biological agent, and that are scalable and capable of rapid development, are needed.

Multiplex PCR, using separate primer sets to produce each amplicon, requires careful design to avoid direct interactions among the primers while providing similar reactivity. See, for example, Caskey, et al., European Patent No. EP 364,255A3; Caskey, et al., U.S. Pat. No. 5,582,989, Wu, et al., U.S. Pat. No. 5,612,473. If the amplification efficiencies are not balanced the product distribution becomes biased. Adding or changing primer sets requires a significant investment in time to ensure the set of primers do not cross-react or produce spurious results. Often sequence analysis is insufficient and empirical optimization is required. Thereafter, several modifications were disclosed, such as the use of inosine nucleotides within primer sequences to better match the amplification reaction efficiency among primers, or the use of chimeric primers in which the target-specific sequence portion was flanked at the 5′ end with a universal sequence portion, again in an effort to make the amplification reaction efficiency more uniform. Nonetheless, these modifications still suffer from the general difficulties of multiplex PCR, such as primer-primer interactions, the need to empirically validate multiplex sets and the increasing difficulty of adding new primer pairs to increase the number of targeted sequences.

More recently a scalable method amenable to high throughput at low cost was disclosed in U.S. Pat. No. 6,858,412 for nucleic acid sequence detection, more particularly for SNP detection and genotyping, which is herein incorporated by reference in its entirety. The method teaches the use of precircle probes that are circularized in the presence of target and later amplified to generate a signal. There remains a need for a cost effective screening methods that provide the minimum level of information necessary to the task such that resources are used in the most efficient manner. Further, a method that can provide different levels of information depending on the need is also desirable.

### SUMMARY OF THE INVENTION

The present invention is directed to methods of using polynucleotide probes to detect the presence or absence of any of several polynucleotide analytes of interest in a sample. The invention is also directed to methods of detecting the presence or absence of each of several polynucleotide analytes in a sample. The invention is further useful for determining, in stages, first whether any of a group of analytes of interest are present in a sample, and if so, in at least one subsequent determination, which of the analytes are present. The invention is also directed towards methods for the rapid and efficient determination of the presence or absence of the members of a particular subset of analytes. The invention also contemplates probes and the corresponding array devices useful for detecting those probes, particularly in a “screening” stage, in which the array comprises array elements equal in number to the number of different tag regions provided in the set of polynucleotide probes. For example, given a probe set of 10,000 probes, each having four distinct tag regions with each tag region comprising one of ten distinct sequences, an array having 40 elements for each distinct sequence of the four tag regions is used to analyze modified probes, instead of, for example, an array with 10,000 array elements.

In one exemplary embodiment for practicing the methods of the invention, a set of polynucleotide probes are prepared, each comprising one or more analyte-specific sequence regions, a common sequence tag, and a reaction site. The analyte-specific region(s) are substantially complementary to sequence region(s) of an analyte of interest, such that the respective probe and target regions will hybridize under hybridization conditions. The common sequence tag is a nucleotide sequence region that is common to a subset of probes, or to all the probes. The probes may further comprise a signature sequence region, wherein the signature sequence is unique within the entire set of probes, or wherein the signature sequence is unique among each set of probes with a particular common sequence tag. The signature sequence tags employed in one particular set of probes with the same common sequence tag may be reused in any or all other sets having a different common sequence tag. Signature sequence tags have a different sequence than any common sequence tag, and are designed to not be homologous to any common sequence tag. Neither the common sequence region nor the signature sequence region is intended to hybridize with target polynucleotide analytes. Each probe also comprises a reaction site at which the probe can be modified, but the modification reaction only proceeds if the probe is hybridized to the target polynucleotide analyte.

In one aspect, the method comprises the steps of (a) mixing together a set of polynucleotide probes with a sample suspected of containing the polynucleotide analytes, (b) after providing conditions and allowing time for substantially complementary probes and analyte targets to hybridize, modifying probes that have hybridized to an analyte, and (c) detecting the common sequence tags present in the modified probes in order to determine the presence or absence in the sample of any of the plurality of polynucleotide analytes. Detection may be accomplished by any of the various methods known to those skilled in the art, including microarray hybridization, hybridization to any solid-phase such as a bead, gel, electrode, container surface, well, etc., molecular beacon, PCR, real-time PCR, TaqMan, hybridization protection assay, sequencing reaction, mass spectrometry, electrophoresis, blotting, sandwich assay and the like. The modified probe may be isolated from the other unmodified prior to performing the detection step.

In one embodiment, there is one common sequence tag among all the probes, and the detection of the common sequence tag (in a modified probe) indicates at least one of the analytes is present. In another embodiment, there are two or more common sequence tags in the probe set representing two or more subsets of probes within the probe set. The detection of any of the common sequence tags indicates at least one of the analytes is present, and the detection of any one particular common sequence tag indicates that at least one analyte targeted by a probe of that subset is present. For example, in a specific embodiment directed towards the detection of pathogens, each probe specific for a different pathogen or different subtype of pathogen (i.e. the subtypes might represent strains of different origin, virulence or drug resistance) would contain the same common sequence tag. Another specific embodiment of a probe set might include two subsets of probes, one subset directed towards pathogens all containing a first common sequence tag, and another subset directed towards non-pathogens all containing a second common sequence tag. In this exemplary embodiment, the detection of the first common sequence is indicative of the presence of a pathogen, while detection of the second common sequence indicates non-pathogenic species are present. Such a determination may usefully serve as a control in the assay.

In another particular embodiment, the probes have two analyte-specific sequence regions, one at each end of a probe. When both regions of a probe are hybridized to the target analyte, the complex formed renders the probe capable of cyclization by either ligation or extension and ligation. In the present invention, the modifying of such a probe comprises cyclization to form a closed circular probe. Following cyclization, the common sequence tag contained in the closed circular probe is detected by direct or indirect methods known in the art. The common sequence tag may also be amplified, by for example rolling circle amplification, and the replicated common sequence tags may then detected by direct or indirect means. In another embodiment, the probes also comprise a cleavage site, by which cyclized probes can be linearized, and then the common sequence tag may be amplified by PCR, TMA, NASBA, CPT, SDA and other nucleic acid amplification methods known in the art. The presence of the common sequence tag may be detected during or following the amplification step.

In another aspect, the method comprises modifying probes hybridized to a target analyte and then in a first stage detecting common sequence tags in the modified probes, and then whenever a positive determination is made, indicating that an analyte is present, detecting the signature sequence tags in the modified probes to determine which of the analytes are present. As appropriate, the detection methods in the first stage and the second stage may differ according to the needs of the operation and/or the convenience of the operator. For example, it may be desirable for the first stage detection process to yield a signal generated in situ, perhaps during or following an amplification reaction such as PCR, in a homogeneous format. Exemplary detection methods include molecular beacons (PHRI, Newark, N.J.), the SMARTCYCLER® system of Cepheid (Sunnyvale, Calif.), TAQMAN® assays of ABI (Foster City, Calif.), and the like, as are commonly known in the art. Or, it may be desirable to generate a signal that can be detected by eye following, for example an ELISA-based assay or a lateral flow assay. Based on whether a positive or negative result is obtained in such a first stage “screening” process, subsequent determinations can be made, and different detections may be more desirable.

In one embodiment, the second detection step is performed by contacting the modified probes under hybridization conditions with a capture probe array, and detecting hybrids formed on the array between the modified probes and the capture probes. In another particular embodiment, the modified probes are first amplified and then the amplicons are contacted with the array to detect the presence of amplified signature sequences. Generally, the capture probes are end-attached to the array surface in a defined pattern, or to the surface of a random array particle. Other multiplex-capable detection methods are also contemplated, using for example mass tags, or electrophoretic tags as are also commonly known in the art.

In yet another aspect, the method comprises the steps of (a) mixing the sample with a probe set under reaction conditions so that probes specifically hybridized to a target are modified to form selectable probes, each selectable probe comprising two common priming sites and a common sequence region there between, (b) isolating the selectable probes from the probes not modified, (c) amplifying the selectable probes, and (d) detecting the amplified probes, so that the presence of any of the plurality of polynucleotide targets is determined. In one particular embodiment, the modified probes are isolated from unmodified probes by enzymatic degradation of the unmodified probes. In this embodiment, modification renders the probes resistant to the action of the enzymatic reaction, thus enabling their isolation. In other embodiments, the modifying step introduces a capture agent in the probe to enable affinity capture of the modified probe followed by a wash step or removal to isolate the modified probes from unmodified probes.

In another aspect, the method comprises including a sequence tag set within a polynucleotide probe such that the sample can be assayed for the presence of any analyte of interest, or the presence of analytes according to subsets of the collection of analytes of interest. The sequence tag set is comprised of two or more tags, each tag being a sequence of approximately 8-40 bases and wherein each tag is represented by one, two, or more unique sequences. Moreover, the sequences representing any particular tag is unique among all the sequences used to represent any tag, and are substantially different from any sequence expected within the analytes of interest.

In one embodiment, a sample is mixed with a set of polynucleotide probes, each probe comprising an analtye-specific sequence region, a unique sequence tag set comprising at least two tags, and a reaction site at which the probe can be modified only when the probe is hybridized to a polynucleotide analyte. Each probe may also further comprise a second analyte-specific region such that the probe is able to form a cyclizable complex upon hybridization to the analyte. After allowing the probes to hybridize to any polynucleotide analyte that may be present, the hybridized probes are modified, and the presence of the members of at least one of the tags in the modified probes is analyzed for, to determine if any of the members of the set of polynucleotide analytes are present. In further step, the modified probes are contacted with an array comprising one spot for each of said tags represented in said sequence tag sets of said set of probes, hybrids are detected, and the pattern of hybridization on the array is read to determine which of said polynucleotide analytes are present, which might be present, and which are absent.

In yet another embodiment, the detection step employs an array comprising elements complementary to each of the unique sequences representing the tags of the sequence tag sets found in the probe set. Thus, for a sequence tag having “n” tags, where each tag has “m” different sequences, the number of elements in the array is equal to m×n, whereas an array that would uniquely identify each possible sequence tag set, and therefore each probe, would have mⁿ elements.

The present invention provides a method for identifying in a sample the presence or absence of any, and/or each, of a plurality of analytes that provides several advantages over existing methods, including, but not limited to (i) rapid one-pot determination of whether any of several pathogens, targets, sequences, mutations and the like are present, (ii) scalable design capable of interrogating analytes numbering from several to several tens of thousands, (iii) signal averaging or signal weighting by e.g. using several probes to target distinct regions of an analyte, (iv) the ability to operate as a two-stage (or multi-stage) screening process in which in a first stage it is determined whether any of the suspected analytes are present, and, in a subsequent stage(s), determining with greater specificity which of the analytes are present, and (v) the ability to chose the level of information assayed for in the sample. Several of these advantages are particularly desirable in cases where the analytes have accessibility or secondary structure issues, as well as for applications that require the rapid analysis of many samples for the presence of many analytes. These advantages are also useful for conducting screening test in which only one or a few hits are expected within a pool of many potential analytes. Accordingly, the invention may be employed, for example, in the screening of bioterror agents; panels of pathogens associated with particular conditions or symptoms, genetically modified organisms, or panels of mutations or polymorphisms associated with particular phenotypes or diseases.

These and other objects and features of the invention will be more fully appreciated when the following detailed description of the invention is read in conjunction with the accompanying drawing and the appended claims.

### BRIEF DESCRIPTION OF THE FIGURES

FIG. 1 is schematic illustration of exemplary methods of modifying a polynucleotide probe in accordance with the invention.

FIGS. 2A-2C are schematic illustrations of exemplary methods of detecting modified probes.

FIGS. 3A-3C are schematic illustrations of exemplary methods of a second stage detection.

FIG. 4 is a schematic illustration of the use of a cyclizable probe for performing an assay.

FIGS. 5A-5B are schematic illustrations of exemplary methods of detecting modified probes in a first stage and a second stage.

FIG. 6 is a schematic illustration of polynucleotide probes with sequence tag sets.

FIGS. 7A-7D are schematic illustrations of arrays useful for detecting modified polynucleotide probes, and representations of exemplary array patterns.

### DETAILED DESCRIPTION OF THE INVENTION

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being, but may also include other organisms including but not limited to mammals, plants, fungi, bacteria or cells derived from any of the above.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication No. WO 99/36760) and PCT/US01/04285 (International Publication No. WO 01/58593).

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 10/442,021, 10/013,598 (U.S. Patent Application Publication 20030036069), and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with hybridization to an array, the sample may be amplified by a variety of mechanisms, some of which may employ PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159, 4,965,188, and 5,333,675. The sample may be amplified on the array. See, for example, U.S. Pat. No. 6,300,070 which is incorporated herein by reference.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245), rolling circle amplification (RCA) (for example, Fire and Xu, PNAS 92:4641 (1995) and Liu et al., J. Am. Chem. Soc. 118:1587 (1996)) and nucleic acid based sequence amplification (NABSA), (See, U.S. Pat. Nos. 5,409,818, 5,554,517, and 6,063,603). Other amplification methods that may be used are described in, U.S. Pat. Nos. 5,242,794, 5,494,810, 4,988,617 and in U.S. Ser. No. 09/854,317. Other amplification methods are also disclosed in Dahl et al., Nuc. Acids Res. 33(8):e71 (2005) and circle to circle amplification (C2CA) Dahl et al., PNAS 101:4548 (2004). Locus specific amplification and representative genome amplification methods may also be used.

Additional methods of sample preparation and techniques for reducing the complexity of a nucleic sample are described in Dong et al., Genome Research 11, 1418 (2001), in U.S. Pat. Nos. 6,872,529, 6,361,947, 6,391,592 and 6,107,023, U.S. Patent Publication Nos. 20030096235 and 20030082543 and U.S. patent application Ser. No. 09/916,135.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, U.S. Ser. No. 10/389,194 and WO99/47964).

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO 99/47964), each of which also is hereby incorporated by reference in its entirety for all purposes. Instruments and software may also be purchased commercially from various sources, including Affymetrix.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/197,621, 10/063,559 (United States Publication No. 20020183936), Ser. Nos. 10/065,856, 10/065,868, 10/328,818, 10/328,872, 10/423,403, and 60/482,389.

A. Definitions

Unless contraindicated or noted otherwise, throughout this specification, the terms “a” and “an” mean one or more, and the term “or” means and/or.

Terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

“Addressable” in reference to a multiplex assay product means that the identity of a probe, e.g. an oligonucleotide tag, or tag complement, protein, peptide, antibody, or the like, can be determined from a spatial location on, or other characteristic of, a solid phase support to which it is attached. In one aspect, an address of a probe is a spatial location, e.g. the planar coordinates of a particular region containing copies of the probe. In other embodiments, probes may be addressed in other ways, e.g. by microparticle size, shape, color, color- or fluorescent ratio, radio frequency of micro-transponder, or the like, e.g. Kettman et al, Cytometry, 33: 234-243 (1998); Xu et al, Nucleic Acids Research, 31: e43 (2003); Pirrung et al., U.S. Pat. No. 6,646,243; Fodor et al., U.S. Pat. No. 6,355,432; Bruchez, Jr. et al, U.S. Pat. No. 6,500,622; Mandecki, U.S. Pat. No. 6,376,187; Stuelpnagel et al, U.S. Pat. No. 6,396,995; Chee et al, U.S. Pat. No. 6,544,732; Chandler et al, PCT publication WO 97/14028; and the like. In one aspect, “addressable” in reference to oligonucleotide probes, oligonucleotide tags, or tag complements means that the nucleotide sequence, or perhaps other physical or chemical characteristics of, an attached probe, such as a tag complement, can be determined from its address, i.e. a one-to-one correspondence exists between the sequence or other property of the attached probe and a spatial location on, or other characteristic of, the solid phase support to which it is attached.

“Allele frequency” in reference to a genetic locus, a sequence marker, or the site of a nucleotide means the frequency of occurrence of a sequence or nucleotide at such genetic locus or the frequency of occurrence of such sequence marker, with respect to a population of individuals. In some contexts, an allele frequency may also refer to the frequency of sequences not identical to, or exactly complementary to, a reference sequence.

“Analyte” is a nucleic acid species whose presence or absence is to be determined by methods described herein. While any particular analyte is simply a nucleic acid species, the meaning or purpose for detecting such nucleic acid species is varied, and includes determining the presence of an organism (e.g. bacteria, virus, fungus, protozoa, animal, etc.), the presence of a particular gene, gene mutation, insertion, deletion, SNP, expressed gene, etc., the presence of a tissue, etc. or via a series of assays the change in the amount of any of the nucleic acid species. Particular forms the analyte may take, and methods of targeting analytes are further discussed below.

“Amplicon” means the product of a polynucleotide amplification reaction. That is, it is a population of polynucleotides, usually double stranded, that are replicated from one or more starting sequences. The one or more starting sequences may be one or more copies of the same sequence, or it may be a mixture of different sequences. Amplicons may be produced by a variety of amplification reactions whose products are multiple replicates of one or more target nucleic acids. Generally, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references that are incorporated herein by reference: Mullis et al, U.S. Pat. Nos. 4,683,195; 4,965,188; 4,683,202; 4,800,159 (PCR); Gelfand et al, U.S. Pat. No. 5,210,015 (real-time PCR with “taqman” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, amplicons of the invention are produced by PCRs. An amplification reaction may be a “real-time” amplification if a detection chemistry is available that permits a reaction product to be measured as the amplification reaction progresses, e.g. “real-time PCR” described below, or “real-time NASBA” as described in Leone et al, Nucleic Acids Research, 26: 2150-2155 (1998), and like references. As used herein, the term “amplifying” means performing an amplification reaction. A “reaction mixture” means a solution containing all the necessary reactants for performing a reaction, which may include, but not be limited to, buffering agents to maintain pH at a selected level during a reaction, salts, co-factors, scavengers, and the like.

“Complementary or substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference. The various hybridization regions, or tags, and primers herein are selected to be “substantially” complementary to their intended hybridization partner. This means that the regions or primers must be sufficiently complementary to hybridize with their respective strands under the given hybridization or polymerization conditions. Therefore, the polynucleotide sequence need not reflect the exact sequence of the complement. For example, a non-complementary nucleotide sequence may be attached to the 5′ end of the primer, with the remainder of the primer sequence being complementary to the strand. Alternatively, non-complementary bases or longer sequences can be interspersed into the region or the primer, provided that the polynucleotide sequence has sufficient complementarity with the sequence of the strand to be hybridized therewith and thereby form a duplex of sufficient stability or structure for the subsequent operation to be performed.

“Complexity” or “complex” in reference to mixtures of nucleic acids means the total length of unique sequences in the mixture. In reference to genomic DNA, complexity means the total length of unique sequence DNA in a genome. The complexity of a genome can be equivalent to or less than the length of a single copy of the genome (i.e. the haploid sequence). Estimates of genome complexity can be less than the total length if adjusted for the presence of repeated sequences. In other words, in reference to genomic DNA, “complexity” means the total number of basepairs present in non-repeating sequences, e.g. Wetmur, Critical Reviews in Biochemistry and Molecular Biology, 26: 227-259 (1991); Britten and Davidson, chapter 1 in Hames et al, editors, Nucleic Acid Hybridization: A Practical Approach (IRL Press, Oxford, 1985).

“Duplex” means at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. In one aspect, stable duplex means that a duplex structure is not destroyed by a stringent wash, e.g. conditions including temperature of about 5° C. less that the Tₘ of a strand of the duplex and low monovalent salt concentration, e.g. less than 0.2 M, or less than 0.1 M. “Perfectly matched” in reference to a duplex means that the poly- or oligonucleotide strands making up the duplex form a double stranded structure with one another such that every nucleotide in each strand undergoes Watson-Crick basepairing with a nucleotide in the other strand. The term “duplex” comprehends the pairing of nucleoside analogs, such as deoxyinosine, nucleosides with 2-aminopurine bases, PNAs, LNAs and the like, that may be employed. A “mismatch” in a duplex between two oligonucleotides or polynucleotides means that a pair of nucleotides in the duplex fails to undergo Watson-Crick bonding.

“Genetic locus,” or “locus” in reference to a genome or target polynucleotide, means a contiguous subregion or segment of the genome or target polynucleotide. As used herein, genetic locus, or locus, may refer to the position of a nucleotide, a gene, or a portion of a gene in a genome, including mitochondrial DNA, or it may refer to any contiguous portion of genomic sequence whether or not it is within, or associated with, a gene. In one aspect, a genetic locus refers to any portion of genomic sequence, including mitochondrial DNA, from a single nucleotide to a segment of few hundred nucleotides, e.g. 100-300, in length. Usually, a particular genetic locus may be identified by its nucleotide sequence, or the nucleotide sequence, or sequences, of one or both adjacent or flanking regions.

“Hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.” “Hybridization conditions” will typically include salt concentrations of less than about 1M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tₘ for the specific sequence at s defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook, Fritsche and Maniatis. “Molecular Cloning A laboratory Manual” 2^{nd} Ed. Cold Spring Harbor Press (1989) and Anderson “Nucleic Acid Hybridization” 1^{st} Ed., BIOS Scientific Publishers Limited (1999), which are hereby incorporated by reference in its entirety for all purposes above. “Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture (e.g., total cellular) DNA or RNA.

“Hybridization-based assay” means any assay that relies on the formation of a stable duplex or triplex between a probe and a target nucleotide sequence for detecting or measuring such a sequence. In one aspect, probes of such assays anneal to (or form duplexes with) regions of target sequences in the range of from 8 to 100 nucleotides; or in other aspects, they anneal to target sequences in the range of from 8 to 40 nucleotides, or more usually, in the range of from 8 to 20 nucleotides. A “probe” in reference to a hybridization-based assay mean a polynucleotide that has a sequence that is capable of forming a stable hybrid (or triplex) with its complement in a target nucleic acid and that is capable of being detected, either directly or indirectly. Hybridization-based assays include, without limitation, assays based on use of oligonucleotides, such as polymerase chain reactions, NASBA reactions, oligonucleotide ligation reactions, single-base extensions of primers, circularizable probe reactions, allele-specific oligonucleotides hybridizations, either in solution phase or bound to solid phase supports, such as microarrays or microbeads. There is extensive guidance in the literature on hybridization-based assays, e.g. Hames et al, editors, Nucleic Acid Hybridization a Practical Approach (IRL Press, Oxford, 1985); Tijssen, Hybridization with Nucleic Acid Probes, Parts I & II (Elsevier Publishing Company, 1993); Hardiman, Microarray Methods and Applications (DNA Press, 2003); Schena, editor, DNA Microarrays a Practical Approach (IRL Press, Oxford, 1999); and the like. In one aspect, hybridization-based assays are solution phase assays; that is, both probes and target sequences hybridize under conditions that are substantially free of surface effects or influences on reaction rate. A solution phase assay may include circumstance where either probes or target sequences are attached to microbeads.

“Kit” refers to any delivery system for delivering materials or reagents for carrying out a method of the invention. In the context of assays, such delivery systems include systems that allow for the storage, transport, or delivery of reaction reagents (e.g. probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials for assays of the invention. In one aspect, kits of the invention comprise probes specific for interfering polymorphic loci. In another aspect, kits comprise nucleic acid standards for validating the performance of probes specific for interfering polymorphic loci. Such contents may be delivered to the intended recipient together or separately. For example, a first container may contain an enzyme for use in an assay, while a second container contains probes.

“Ligation” means to form a covalent bond or linkage between the termini of two or more nucleic acids, e.g. oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon of a terminal nucleotide of one oligonucleotide with 3′ carbon of another oligonucleotide. A variety of template-driven ligation reactions are described in the following references, which are incorporated by reference: Whitely et al, U.S. Pat. No. 4,883,750; Letsinger et al, U.S. Pat. No. 5,476,930; Fung et al, U.S. Pat. No. 5,593,826; Kool, U.S. Pat. No. 5,426,180; Landegren et al, U.S. Pat. No. 5,871,921; Xu and Kool, Nucleic Acids Research, 27: 875-881 (1999); Higgins et al, Methods in Enzymology, 68: 50-71 (1979); Engler et al, The Enzymes, 15: 3-29 (1982); and Namsaraev, U.S. patent publication 2004/0110213.

“Microarray” or “array” refers to a solid phase support having a planar surface, which carries an array of nucleic acids, each member of the array comprising identical copies of an oligonucleotide or polynucleotide immobilized to a spatially defined region or site, which does not overlap with those of other members of the array; that is, the regions or sites are spatially discrete. Spatially defined hybridization sites may additionally be “addressable” in that its location and the identity of its immobilized oligonucleotide are known or predetermined, for example, prior to its use. Ordered arrays include, but are not limited to, those prepared by photolithography, spotting, printing, electrode arrays, “gel pad” arrays, and the like. The size of array can vary from one element to thousands, tens of thousands, or even millions of elements. Depending on the number of array elements required, some array types or methods of preparing the array may be more advantageous, as those skilled in the art are aware. Typically, the oligonucleotides or polynucleotides are single stranded and are covalently attached to the solid phase support, usually by a 5′-end or a 3′-end. The density of non-overlapping regions containing nucleic acids in a microarray is typically greater than 100 per cm², and more preferably, greater than 1000 per cm². Microarray technology is reviewed in the following references: Schena, Editor, Microarrays: A Practical Approach (IRL Press, Oxford, 2000); Southern, Current Opin. Chem. Biol., 2: 404-410 (1998); Nature Genetics Supplement, 21: 1-60 (1999). As used herein “microarray” or “array” may also refer to a “random microarray” or “random array”, which refer to an array whose spatially discrete regions of oligonucleotides or polynucleotides are not spatially addressed. That is, the identity of the attached oligonucleotides or polynucleotides is not discernable, at least initially, from its location. In one aspect, random microarrays are planar arrays of microbeads wherein each microbead has attached a single kind of hybridization tag complement, such as from a minimally cross-hybridizing set of oligonucleotides. Arrays of microbeads may be formed in a variety of ways, e.g. Brenner et al, Nature Biotechnology, 18: 630-634 (2000); Tulley et al, U.S. Pat. No. 6,133,043; Pirrung et al., U.S. Pat. No. 6,646,243; Fodor et al., U.S. Pat. No. 6,355,432; Stuelpnagel et al, U.S. Pat. No. 6,396,995; Chee et al, U.S. Pat. No. 6,544,732; and the like. Likewise, after formation, microbeads, or oligonucleotides thereof, in a random array may be identified in a variety of ways, including by optical labels, e.g. fluorescent dye ratios or quantum dots, shape, sequence analysis, or the like.

“Modified probe” as used herein refers to a polynucleotide probe that has been changed in structure, sequence and/or composition as the result of a reaction that is dependent upon the probe having hybridized to a particular polynucleotide sequence (“targeted sequence”) in an analyte.

“Nucleoside” as used herein includes the natural nucleosides, including 2′-deoxy and 2′-hydroxyl forms, e.g. as described in Kornberg and Baker, DNA Replication, 2nd Ed. (Freeman, San Francisco, 1992). “Analogs” in reference to nucleosides includes synthetic nucleosides having modified base moieties and/or modified sugar moieties, e.g. described by Scheit, Nucleotide Analogs (John Wiley, New York, 1980); Uhlman and Peyman, Chemical Reviews, 90: 543-584 (1990), or the like, with the proviso that they are capable of specific hybridization. Such analogs include synthetic nucleosides designed to enhance binding properties, reduce complexity, increase specificity, and the like. Polynucleotides comprising analogs with enhanced hybridization or nuclease resistance properties are described in Uhlman and Peyman (cited above); Crooke et al, Exp. Opin. Ther. Patents, 6: 855-870 (1996); Mesmaeker et al, Current Opinion in Structural Biology, 5: 343-355 (1995); and the like. Exemplary types of polynucleotides that are capable of enhancing duplex stability include oligonucleotide N3′→P5′ phosphoramidates (referred to herein as “amidates”), peptide nucleic acids (referred to herein as “PNAs”), oligo-2′-O-alkylribonucleotides, polynucleotides containing C-5 propynylpyrimidines, locked nucleic acids (LNAs), and like compounds. Such oligonucleotides are either available commercially or may be synthesized using methods described in the literature.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature>90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g. 200 nL, to a few hundred μL, e.g. 200 μL. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“taqman”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al, Anal. Biochem., 273: 221-228 (1999)(two-color real-time PCR). Usually, distinct sets of primers are employed for each sequence being amplified. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β₂-microglobulin, ribosomal RNA, and the like. Techniques for quantitative PCR are well-known to those of ordinary skill in the art, as exemplified in the following references that are incorporated by reference: Freeman et al, Biotechniques, 26: 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); Becker-Andre et al, Nucleic Acids Research, 17: 9437-9446 (1989); and the like.

“Polymorphism” or “genetic variant” means a substitution, inversion, insertion, or deletion of one or more nucleotides at a genetic locus, or a translocation of DNA from one genetic locus to another genetic locus. In one aspect, polymorphism means one of multiple alternative nucleotide sequences that may be present at a genetic locus of an individual and that may comprise a nucleotide substitution, insertion, or deletion with respect to other sequences at the same locus in the same individual, or other individuals within a population. An individual may be homozygous or heterozygous at a genetic locus; that is, an individual may have the same nucleotide sequence in both alleles, or have a different nucleotide sequence in each allele, respectively. In one aspect, insertions or deletions at a genetic locus comprises the addition or the absence of from 1 to 10 nucleotides at such locus, in comparison with the same locus in another individual of a population (or another allele in the same individual. Usually, insertions or deletions are with respect to a major allele at a locus within a population, e.g. an allele present in a population at a frequency of fifty percent or greater.

“Polynucleotide” or “oligonucleotide” are used interchangeably and each mean a linear polymer of nucleotide monomers. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g. naturally occurring or non-naturally occurring analogs. Non-naturally occurring analogs may include PNAs, LNAs, phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moities, or bases at any or some positions. Polynucleotides typically range in size from a few monomeric units, e.g. 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or internucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references.

“Primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Usually primers are extended by a DNA polymerase. Primers usually have a length in the range of from 14 to 36 nucleotides.

“Readout” means a characteristic of one or more signal generation moieties, or labels, that are measured, detected, and/or counted and that can be converted to a number or value. In one aspect, a readout of an assay is obtained by the use or application of a instrument and/or process that converts assay results on the molecular level into signals that may be detected and recorded. Such instrument or process may be referred to as a “readout device” (or instrument) or “readout process” (or method). A readout can also include, or refer to, an actual numerical representation of such collected or recorded data. For example, a readout of a hybridization assay using a microarray as a readout device collectively refers to signals generated at each feature, or hybridization site, of the microarray and their numerical, graphical, and/or pictorial representations.

“Sample” means a quantity of material from a biological, environmental, medical, or patient source in which detection or measurement of target nucleic acids is sought. On the one hand it is meant to include a specimen or culture (e.g., microbiological cultures). On the other hand, it is meant to include both biological and environmental samples. A sample may include a specimen of synthetic origin. Biological samples may be animal, including human, fluid, solid (e.g., stool) or tissue, as well as liquid and solid food and feed products and ingredients such as dairy items, vegetables, meat and meat by-products, and waste. Biological samples may include materials taken from a patient including, but not limited to cultures, blood, saliva, cerebral spinal fluid, pleural fluid, milk, lymph, sputum, semen, needle aspirates, and the like. Biological samples may be obtained from all of the various families of domestic animals, as well as feral or wild animals, including, but not limited to, such animals as ungulates, bear, fish, rodents, etc. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention. “Sample” is also used to refer to the solution derived from any of the above sources as it processed in preparation for further testing or assays.

“Solid support”, “support”, and “solid phase support” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. Microarrays usually comprise at least one planar solid phase support, such as a glass microscope slide.

“Specific” or “specificity” in reference to the binding of one molecule to another molecule, such as a labeled target sequence for a probe, means the recognition, contact, and formation of a stable complex between the two molecules, together with substantially less recognition, contact, or complex formation of that molecule with other molecules. In one aspect, “specific” in reference to the binding of a first molecule to a second molecule means that to the extent the first molecule recognizes and forms a complex with another molecules in a reaction or sample, it forms the largest number of the complexes with the second molecule. Preferably, this largest number is at least fifty percent. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like. As used herein, “contact” in reference to specificity or specific binding means two molecules are close enough that weak non-covalent chemical interactions, such as Van der Waal forces, hydrogen bonding, base-stacking interactions, ionic and hydrophobic interactions, and the like, dominate the interaction of the molecules.

“Targeting sequence” refers to that portion or region of a polynucleotide probe that is designed to be substantially complementary to a sequence found in an analyte. The “targeting sequence” is also synonymously referred to as the “analyte-specific region” of the probe. Conversely, the sequence in the analyte is referred to as the “targeted sequence”.

“Tₘ” is used in reference to “melting temperature.” Melting temperature is the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation. Tₘ=81.5+0.41 (% G+C), when a nucleic acid is in aqueous solution at 1 M NaCl (see e.g., Anderson and Young, Quantitative Filter Hybridization, in Nucleic Acid Hybridization (1985). Other references (e.g., Allawi, H. T. & SantaLucia, J., Jr., Biochemistry 36, 10581-94 (1997)) include alternative methods of computation which take structural and environmental, as well as sequence characteristics into account for the calculation of Tₘ.

B. Polynucleotide Analytes

The methods of the invention are generally directed towards the detection of polynucleotide sequences, and such sequences of interest may be derived from any and all types of samples discussed above and may originate from prokaryotes, eukaryotes, viruses, protozoa or any chimera or construct assembled by man. Any types of nucleic acid molecules may be analyzed or assayed for using the methods of the present invention, such as DNA, RNA, mitochondrial DNA, rRNA, mRNA, tRNA, etc.

What particularly is the “analyte” depends upon the purpose of the assay and the type of information that is to be obtained. The analyte may be a sequence that identifies a species. As such, any unique, identifying sequence within such species may be designated and used as the analyte. The analyte may be a sequence that identifies a gene. Similarly, an identifying sequence within the gene (intron, exon, or untranslated regions, etc.) may be used as the analyte. The same holds for any type of target of interest, such as, for example, mutations or polymorphs within genes, expressed sequences, sex-linked chromosomes, insertions and deletions, genetic modifications (found in genetically engineered organisms) and the like. Different analytes may be located in close proximity to one another along the same polynucleotide strand. Thus in some instances it may be more accurate to consider each targeted sequence as the analyte rather than a particular strand, or gene, or construct as the analyte. Conversely, in some instances, multiple probes targeting different sequences might be used to detect the presence of a particular analyte. For example, where the purpose is to detect a particular species, it may be desirable to target different portions of the organism's DNA/RNA in order to achieve greater sensitivity. The signal from the multiple probes will be larger than that from a single probe, but note that the analyte of interest in this case is polynucleotide sequence indicative of the species, and that it is embodied in this instance by multiple different targeted sequences.

Standard methods for the preparation of samples for analysis, such as for example the isolation, concentration, fragmentation, or denaturation of the nucleic acid target, are to be employed as required, and generally follow the methods known in the art of nucleic acid analysis. Methods practiced by the forensic sciences are also contemplated for the use, care and handling of small sample amounts, samples to be extracted from other materials, and samples that have aged. Also contemplated are methods for sampling large volumes of air samples, for airborne contaminants and pathogens.

C. Polynucleotide Probes

1. Probe Regions

The probes used in practicing the methods of the invention are comprised of several regions or tags. Some of these regions are introduced here with regard to their general structure, and a more detailed explanation of their role and operation are provided below with reference to the figures.

a. Analyte-Specific Region

Generally, probes of the invention specifically hybridize to their corresponding target polynucleotide at their analyte-specific region, which has a length in the range of from 9 to 100 nucleotides, more typically in the range of from 15 to 40 nucleotides. The analyte-specific region is substantially complementary to the targeted region of the analyte, preferably the two sequences are perfectly matched. Usually, such targeted region on the analyte is contiguous, although loops may form within the targeted region. In some embodiments, a probe may have two (or more) different analyte-specific regions and bind to two (or more) non-continuous regions of the analyte, e.g. as with gap-ligation probes, as disclosed in Abravaya et al., Nucleic Acids Research, 23: 675-682 (1995); or in Hardenbol et al (cited above).

b. Sequence Tags and Sequence Regions

“Sequence tag” and “sequence region” are used interchangeably and generally refer to a contiguous polynucleotide region within a probe that are used for specific hybridization operations. Several different types of regions or tags are employed in the methods of the invention. “Common sequence tag” (CST) refers to region within a probe that is a member of a probe set that is used to identify that probe has a member of that set. “Signature sequence tag” (SST) refers to a region within a probe that is a member of a probe set that is used to distinguish and identify that probe from the other members of the probe set. “Sequence tag set” (STS) refers to a set of neighboring polynucleotide regions (each region being a “tag”; see below), which may be contiguous or separating by one or several nucleotides, that form an encoded signature for each probe of a probe set.

The sequence tags generally have a length of from 9 to 100 bases, more generally in the range of from 15 to 40 bases. The length is not critical per se, other than to ensure that the regions can specifically hybridize to the intended target and that the sequences are sufficiently unique and non-homologous to those of other probes and non-targeted sequences that cross-hybridization and non-specific hybridization is not a factor in the operation of the methods.

c. Designing the Probe Regions: Sequence Tags and Minimally Cross-Hybridizing Sets.

In one aspect, the invention employs minimally cross-hybridizing sets of “sequence tags”, such as disclosed in Brenner et al, U.S. Pat. No. 5,846,719; Mao et al (cited above); Fan et al, International patent publication WO 2000/058516; Morris et al, U.S. Pat. No. 6,458,530; Morris et al, U.S. patent publication 2003/0104436; Church et al, European patent publication 0 303 459; Huang et al, U.S. Pat. No. 6,709,816; which references are incorporated herein by reference. The sequences of polynucleotides of a minimally cross-hybridizing set differ from the sequences of every other member of the same set by at least two nucleotides, and more preferably, by at least three nucleotides. Thus, each member of such a set cannot form a duplex (or triplex) with the complement of any other member with less than two mismatches, or three mismatches as the case may be. Preferably, perfectly matched duplexes of tags and tag complements of the same minimally cross-hybridizing set have approximately the same stability, especially as measured by melting temperature. Complements of oligonucleotide tags, referred to herein as “tag complements,” may comprise natural nucleotides or non-natural nucleotide analogs. In one aspect, non-natural nucleic acid analogs are used as tag complements that remain stable under repeated washings and hybridizations of oligonucleotide tags. In particular, tag complements may comprise peptide nucleic acids (PNAs) or LNAs. Oligonucleotide tags from the same minimally cross-hybridizing set when used with their corresponding tag complements provide a means of enhancing specificity of hybridization. Microarrays of tag complements are available commercially, e.g. GenFlex Tag Array (Affymetrix, Santa Clara, Calif.); and their construction and use are disclosed in Fan et al, International patent publication WO 2000/058516; Morris et al, U.S. Pat. No. 6,458,530; Morris et al, U.S. patent publication 2003/0104436; and Huang et al U.S. Pat. No. 6,709,816.

As mentioned above, in one aspect tag complements comprise PNAs, which may be synthesized using methods disclosed in the art, such as Nielsen and Egholm (eds.), Peptide Nucleic Acids: Protocols and Applications (Horizon Scientific Press, Wymondham, UK, 1999); Matysiak et al, Biotechniques, 31: 896-904 (2001); Awasthi et al, Comb. Chem. High Throughput Screen., 5: 253-259 (2002); Nielsen et al, U.S. Pat. No. 5,773,571; Nielsen et al, U.S. Pat. No. 5,766,855; Nielsen et al, U.S. Pat. No. 5,736,336; Nielsen et al, U.S. Pat. No. 5,714,331; Nielsen et al, U.S. Pat. No. 5,539,082; and the like, which references are incorporated herein by reference. Construction and use of microarrays comprising PNA tag complements are disclosed in Brandt et al, Nucleic Acids Research, 31(19), e119 (2003).

Preferably, oligonucleotide tags and tag complements are selected to have similar duplex or triplex stabilities to one another so that perfectly matched hybrids have similar or substantially identical melting temperatures. This permits mismatched tag complements to be more readily distinguished from perfectly matched tag complements in the hybridization steps, e.g. by washing under stringent conditions. Guidance for carrying out such selections is provided by published techniques for selecting optimal PCR primers and calculating duplex stabilities, e.g. Rychlik et al, Nucleic Acids Research, 17: 8543-8551 (1989) and 18: 6409-6412 (1990); Breslauer et al, Proc. Natl. Acad. Sci., 83: 3746-3750 (1986); Wetmur, Crit. Rev. Biochem. Mol. Biol., 26: 227-259 (1991); and the like. A minimally cross-hybridizing set of oligonucleotides can be screened by additional criteria, such as GC-content, distribution of mismatches, theoretical melting temperature, and the like, to form a subset which is also a minimally cross-hybridizing set.

2. Preparation of Probes

Probes of the invention comprise oligonucleotides that are made by conventional methodologies, e.g. by direct synthesis, or for longer probes, convergent synthesis, e.g. as disclosed in Namsaraev, U.S. patent publication 2004/0110213, which is incorporated herein by reference. However, it must be recognized that many variations to a natural nucleic acid structure can be introduced using such conventional methodologies.

3. Modifying Probes

Modified probes are formed in a reaction that is dependent upon the probe first specifically hybridizing to a target polynucleotide. In one aspect, such specific hybridization creates a substrate for an enzyme that modifies the probe, either to implement a first step in conversion to a selectable probe, or to bring about a complete conversion to a selectable probe. Such modification in whole or in part confers a property on the modified probe that allows it to be selected, or removed from, unmodified probes. For example, such selection may be effected by removal or separation from unmodified probes, by destruction of unmodified probes, or by other such means. Modifications may be carried out chemically or enzymatically.

Usually, probes are modified enzymatically, such as by ligation, extension with a polymerase, or the like. In one aspect, probes are modified by ligation so that they form closed circular DNAs. In another aspect, probes are extended by a nucleic acid polymerase to incorporate a modified nucleotide that contains a capture moiety, such as biotin, or that contains a label agent. In another aspect, both of the above modifications are accomplished by one or more template-driven enzymatic reactions. Exemplary probes include molecular inversion probes, padlock probes, rolling circle probes, ligation-based probes with “zip-code” tags, single-base extension probes, and the like, e.g. Hardenbol et al, Nature Biotechnology, 21: 673-678 (2003); Nilsson et al, Science, 265: 2085-2088 (1994); Baner et al, Nucleic Acids Research, 26: 5073-5078 (1998); Lizardi et al, Nat. Genet., 19: 225-232 (1998); Gerry et al, J. Mol. Biol., 292: 251-262 (1999); Fan et al, Genome Research, 10: 853-860 (2000); International patent publications WO 2002/57491 and WO 2000/58516; U.S. Pat. Nos. 6,506,594 and 4,883,750; and the like, which references are incorporated herein by reference.

In one aspect, probes of the invention are molecular inversion probes, e.g. as disclosed in Hardenbol et al (cited above) and in Willis et al, International patent publication WO 2002/057491. In the case of molecular inversion probes, selectable probes are formed by circularizing probes in a template-driven reaction on a target polynucleotide followed by digestion of non-circularized polynucleotides, such as target polynucleotides, unligated probe, probe concatamers, and the like, with an exonuclease. In another aspect, probes of the invention comprise an oligonucleotide tag and a target-specific region that is extended by a polymerase reaction to add a nucleotide with a capture moiety, such as biotin, as disclosed in Fan et al (cited above) and Mao et al (cited above). Such modified probes are captured on a solid phase support derivatized with a binding partner, e.g. avidin-coated magnetic microbeads, and then separated from the unmodified probes in the reaction mixture.

Many different terminator-capture moiety combinations are available. Preferably, dideoxynucleoside triphosphates are used as terminators. In one aspect, capture moieties may be attached to such terminators derivatized with an alkynylamino group, as taught by Hobbs et al, U.S. Pat. No. 5,047,519 and Taing et al, International patent publication WO 02/30944, which are incorporated herein by reference. Preferable capture moieties include biotin or biotin derivatives, such as desthiobiotin, which are captured with streptavidin or avidin or commercially available antibodies, and dinitrophenol, digoxigenin, fluorescein, and rhodamine, all of which are available as NHS-esters that may be reacted with alkynylamino-derivatized terminators. These reagents as well as antibody capture agents for these compounds are available from, e.g. Molecular Probes, Inc. (Eugene, Oreg.).

D. Operations Used in the Method

1. Hybridization-Based Assays

As mentioned above, the invention relates in some aspects to the use of hybridization-based operations to detect or measure a plurality of analytes. Hybridization-based assays are widely used in multiplexed formats to simultaneously genotype DNA samples at multiple loci, e.g. allele-specific multiplex PCR, arrayed primer extension (APEX) technology, variation detection arrays, solution phase primer extension or ligation assays, and the like, described in the following references: Shumaker et al, Hum. Mut., 7: 346-354 (1996); Cronin et al, U.S. Pat. No. 6,468,744; Huang et al, U.S. Pat. Nos. 6,709,816 and 6,287,778; Fan et al, U.S. patent publication 2003/0003490; Chee et al, U.S. Pat. No. 6,355,431; Gunderson et al, U.S. patent publication 2005/0037393; Hacia et al, U.S. Pat. No. 6,342,355; Kennedy et al, Nature Biotechnology, 21: 1233-1237 (2003); Chou et al, Clin. Chem., 49: 542-551 (2003); and the like.

In one aspect, hybridization-based assays include circularizing probes, such as padlock probes, rolling circle probes, molecular inversion probes, linear amplification molecules for multiplexed PCR, and the like, e.g. padlock probes being disclosed in U.S. Pat. Nos. 5,871,921; 6,235,472; 5,866,337; and Japanese patent JP 4-262799; rolling circle probes being disclosed in Aono et al, JP-4-262799; Lizardi, U.S. Pat. Nos. 5,854,033; 6,183,960; 6,344,239; molecular inversion probes being disclosed in Hardenbol et al (cited above) and in Willis et al, U.S. patent publication 2004/0101835; and linear amplification molecules being disclosed in Faham et al, U.S. patent publication 2003/0104459; all of which are incorporated herein by reference. Such probes are desirable because non-circularized probes can be digested with single stranded exonucleases thereby greatly reducing background noise due to spurious amplifications, and the like. In the case of molecular inversion probes (MIPs), padlock probes, and rolling circle probes, constructs for generating labeled target sequences are formed by circularizing a linear version of the probe in a template-driven reaction on a target polynucleotide followed by digestion of non-circularized polynucleotides in the reaction mixture, such as target polynucleotides, unligated probe, probe concatamers, and the like, with an exonuclease, such as exonuclease I.

2. Labeling Oligonucleotide Tags

Modified probes and the sequence tags generated in accordance with the invention can be labeled in a variety of ways, including the direct or indirect attachment of fluorescent moieties, calorimetric moieties, chemiluminescent moieties, and the like. Many comprehensive reviews of methodologies for labeling DNA provide guidance applicable to generating labeled oligonucleotide tags of the present invention. Such reviews include Haugland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); Wetmur, Critical Reviews in Biochemistry and Molecular Biology, 26: 227-259 (1991); and the like. Particular methodologies applicable to the invention are disclosed in the following sample of references: Fung et al, U.S. Pat. No. 4,757,141; Hobbs, Jr., et al U.S. Pat. No. 5,151,507; Cruickshank, U.S. Pat. No. 5,091,519. In one aspect, one or more fluorescent dyes are used as labels for the oligonucleotide tags, e.g. as disclosed by Menchen et al, U.S. Pat. No. 5,188,934 (4,7-dichlorofluorscein dyes); Begot et al, U.S. Pat. No. 5,366,860 (spectrally resolvable rhodamine dyes); Lee et al, U.S. Pat. No. 5,847,162 (4,7-dichlororhodamine dyes); Khanna et al, U.S. Pat. No. 4,318,846 (ether-substituted fluorescein dyes); Lee et al, U.S. Pat. No. 5,800,996 (energy transfer dyes); Lee et al, U.S. Pat. No. 5,066,580 (xanthene dyes): Mathies et al, U.S. Pat. No. 5,688,648 (energy transfer dyes); and the like. As used herein, the term “fluorescent signal generating moiety” means a signaling means which conveys information through the fluorescent absorption and/or emission properties of one or more molecules. Such fluorescent properties include fluorescence intensity, fluorescence life time, emission spectrum characteristics, energy transfer, and the like. In particular, many schemes for generating copies of labeled oligonucleotide tags for hybridization to microarrays are disclosed in Namsaraev et al, International patent publication WO 2005/029040, which is incorporated herein by reference.

3. Hybridization of Modified Probes/Sequence Tags to Solid Supports

Methods for hybridizing labeled oligonucleotide tags to microarrays, and like platforms, suitable for the present invention are well known in the art. Guidance for selecting conditions and materials for applying labeled target sequences to solid phase supports, such as microarrays, may be found in the literature, e.g. Wetmur, Crit. Rev. Biochem. Mol. Biol., 26: 227-259 (1991); Chee et al, Science, 274: 610-614 (1996); Duggan et al, Nature Genetics, 21: 10-14 (1999); Schena, Editor, Microarrays: A Practical Approach (IRL Press, Washington, 2000); Freeman et al, Biotechniques, 29: 1042-1055 (2000); and like references. Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference. Hybridization conditions typically include salt concentrations of less than about 1M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will stably hybridize to a perfectly complementary target sequence, but will not stably hybridize to sequences that have one or more mismatches. The stringency of hybridization conditions depends on several factors, such as probe sequence, probe length, temperature, salt concentration, concentration of organic solvents, such as formamide, and the like. How such factors are selected is usually a matter of design choice to one of ordinary skill in the art for any particular embodiment. Usually, stringent conditions are selected to be about 5° C. lower than the Tₘ for the specific sequence for particular ionic strength and pH. Exemplary hybridization conditions include salt concentration of at least 0.01 M to no more than 1 M Na ion concentration (or other salts) at a pH 7.0 to 8.3 and a temperature of at least 25° C. Additional exemplary hybridization conditions include the following: 5×SSPE (750 mM NaCl, 50 mM sodium phosphate, 5 mM EDTA, pH 7.4).

Exemplary hybridization procedures for applying labeled oligonucleotide tags to a GENFLEX microarray (Affymetrix, Santa Clara, Calif.) is as follows: denatured labeled target sequence at 95-100° C. for 10 minutes and snap cool on ice for 2-5 minutes. The microarray is pre-hybridized with 6×SSPE-T (0.9 M NaCl 60 mM NaH₂, PO₄, 6 mM EDTA (pH 7.4), 0.005% Triton X-100)+0.5 mg/ml of BSA for a few minutes, then hybridized with 120 μL hybridization solution (as described below) at 42° C. for 2 hours on a rotisserie, at 40 RPM. Hybridization Solution consists of 3M TMACL (tetramethylammonium. chloride), 50 mM MES ((2-[N-morpholino]ethanesulfonic acid) sodium salt) (pH 6.7), 0.01% of Triton X-100, 0.1 mg/ml of Herring Sperm DNA, optionally 50 pM of fluorescein-labeled control oligonucleotide, 0.5 mg/ml of BSA (Sigma) and labeled target sequences in a total reaction volume of about 120 μL. The microarray is rinsed twice with 1×SSPE-T for about 10 seconds at room temperature, then washed with 1×SSPE-T for 15-20 minutes at 40° C. on a rotisserie, at 40 RPM. The microarray is then washed 10 times with 6×SSPE-T at 22° C. on a fluidic station (e.g. model FS400, Affymetrix, Santa Clara, Calif.). Further processing steps may be required depending on the nature of the label(s) employed, e.g. direct or indirect. Microarrays containing labeled target sequences may be scanned on a confocal scanner (such as available commercially from Affymetrix) with a resolution of 60-70 pixels per feature and filters and other settings as appropriate for the labels employed. GeneChip Software (Affymetrix) may be used to convert the image files into digitized files for further data analysis.

4. Detection of Hybridized Labeled Tag Sequences

Labeled oligonucleotide tags of the invention are detected by specifically hybridizing them to one or more solid supports containing end-attached tag complements, usually in the form of a microarray of spatially discrete hybridization sites. Instruments for measuring optical signals, especially fluorescent signals, from labeled tags hybridized to targets on a microarray are described in the following references which are incorporated by reference: Stern et al, PCT publication WO 95/22058; Resnick et al, U.S. Pat. No. 4,125,828; Karnaukhov et al, U.S. Pat. No. 4,354,114; Trulson et al, U.S. Pat. No. 5,578,832; Pallas et al, PCT publication WO 98/53300; and the like.

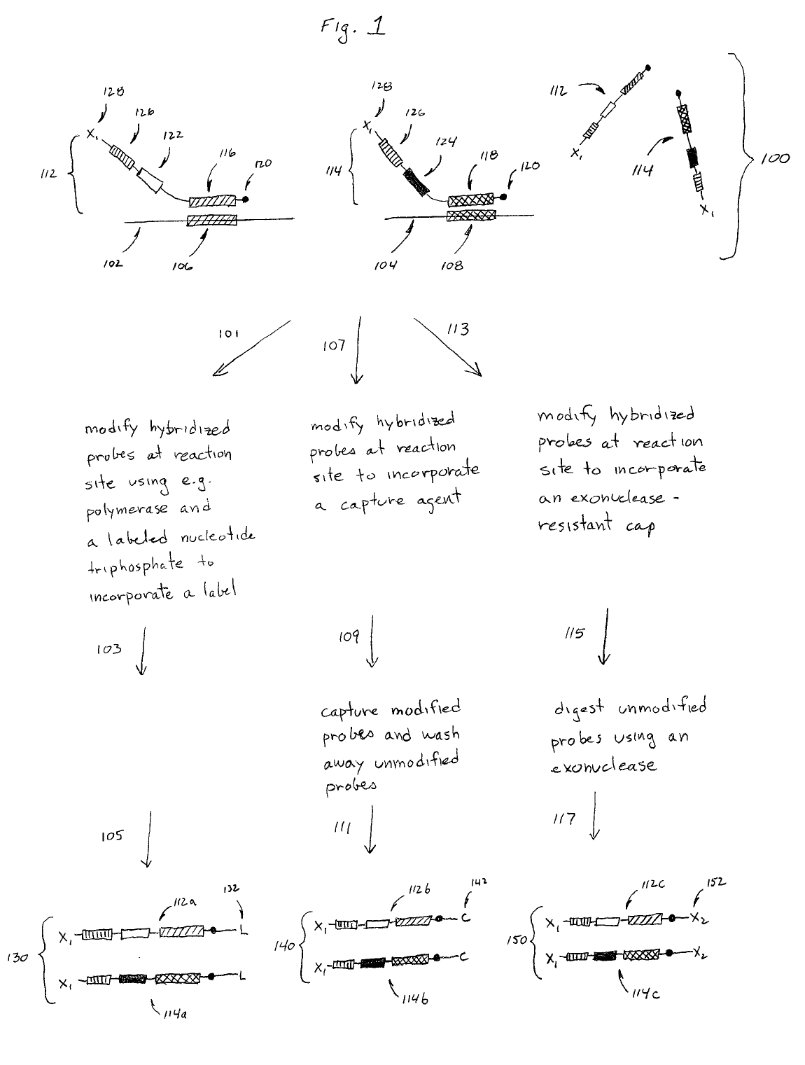

The invention shall now be further described with reference to the figures. FIG. 1 illustrates several methods by which, in one aspect of the invention, a probe set is mixed with sample suspected of containing target analytes, the probes are modified as the result of reactions that occur based on the presence of analyte(s), and any modified probes produced are available for detection. In a solution 100, polynucleotide probes 112 and 114 are mixed with a sample containing analytes 102 and 104. The conditions of solution 100, such as temperature, ion composition and ionic strength, volume exclusion agents, oligonucleotide promoters, etc. are adjusted, if necessary to support hybridization between probe and analyte. Hybrid formation occurs between the targeted sequence 106 and 108 of analytes 102 and 104, respectively, and the analyte-specific region 116 and 118 of probes 112 and 114, respectively. The analyte-specific regions are sequences designed to be at least substantially complementary to the intended target sequence of each analyte of interest. In a preferred embodiment the analyte-specific regions are completely complementary over their length to a portion of the target sequence. For example, if the analyte-specific region is 20 bases there is a corresponding region in the target that is the 20 base complement of the analyte-specific region.

In the probe sets illustrated in FIG. 1, each probe has one analyte-specific region, although there may be more than one (i.e. non-contiguous) analyte-specific regions in a probe. The length of an analyte-specific region is typically at least five bases, more typically at least 15 bases and may be up to about 100 bases. Because the number of bases roughly determines the energetics of the binding interaction, the probes are designed, according to principles well known in the art for designing hybridization probes and primers, in consideration of the desired operating temperatures, conditions and specificity. The length is also set with regard to the desired method and yield of synthesizing the probe. The various probes of a set may have the same or different lengths of an analyte-specific region, and the probe-analyte duplexes may have different Tₘ and T_{d} properties. However it is desirable to design the probes of a set to at least have similar properties (e.g. length, Tₘ, etc.) such that the dynamics and conditions used for hybridizing probes to analytes are similar, and thus the operation will yield specifically hybridized probes for a given operating method. Moreover, it is desirable to have probe sets that have similar properties and thus similar operating conditions so that standardized manual or automated protocols can be used in performing the methods.

Probes 112 and 114 also comprise a reaction site 120, a common sequence tag 126, and signature sequences 122 and 124, respectively. The probes 112 and 114 also optionally comprise a capping group 128, denoted as X₁, that resists enzymatic degradation, particularly by exonucleases, and thus prevents enzymatic degradation of the probe. The length of the common sequence tag is preferably 15 to 30 bases and most preferably 20 to 25 bases. The length of the signature sequence is preferably 15 to 30 bases and most preferably 20 to 25 bases. In some embodiments the signature sequence is optional.

The reaction site 120 is a functional group present in the probe to which another reactant can chemically bond. Both enzymatic and chemical reactions are contemplated for use in any of the methods. Where the method does not include an amplification step (see below) the reaction does not need to produce an amplifiable structure and therefore non-natural structures can be tolerated. However, where amplification is to be performed the structure of the modified probe, and particularly the structure immediate around the reaction site, should be amplifiable, and therefore should be a natural polynucleotide structure. Typically the reaction site will be 3′- or 5′-hydroxyl group of a ribose or deoxyribose subunit, or a phosphate group on either a 3′- or 5′-hydroxyl group. These natural structures are particularly favored where the modifying reaction is an enzymatic reaction.

Common sequence tag 126 is present in each of the probes of a collection of probes (probes 112 and 114 in FIG. 1). In contrast, signature sequence tags 122 and 124 are unique, respectively, to probes 112 and 114. The common and signature tags may be present in any order or orientation with respect to one another, and they may be contiguous, separated by 1, 5, 10 or more bases, base analogs, spacers and the like.

In operation, the probes are mixed with a sample suspected of containing analytes and the conditions are adjusted to promote hybridization between the analyte-specific regions 116 and 118 of the probes and the analytes. The probes are typically added in an amount in excess of the amount of analyte suspected, as is commonly known in the art of hybridization assays. Some probes will not have an analyte partner with which to hybridize. In some instances an analyte will not be present, and none of the corresponding probes will become hybridized. Probes that are hybridized to an analyte are modified after hybridization.

Three scenarios for modifying probes are illustrated within FIG. 1. For example, in step 101, hybridized probes are modified in a templated extension reaction using, e.g. a DNA polymerase enzyme and a set of dNTPs in which at least one of the dNTPs includes a label agent 132. The dNTPs are added to the probe at the reaction site, and the extension product will include an incorporated label agent 132. Terminators may optionally be included, but with due consideration given to the manner of label incorporation. The terminator itself may include the label agent 132. Preferably, only one type of label is used in the modifying step. I some instances, the sample may be split and separate modification reactions performed such as for single base extension reactions or other polymorphism detection methods. Where the sample is split and separate modifying reactions are performed, the labels may be the same or different in each reaction. If the labels are the same the subsequent detection step will be done separately, but if the labels are different the samples may be again combined and assessed by multiplexed detection techniques (e.g. multi-color fluorescent detection). The result of the reaction is to produce (105) a set 130 of modified probes 112a and 114a, that have been modified with a label agent 132 when the corresponding analyte is present in the sample. The modified probes are detectable via the label agent 132, whereas any unmodified probes will not be detected because they lack a label agent 132.

In step 107, the process is similar to that of step 101, except that the modification reaction produces a set of probes that have been modified to contain a capture agent 142. Typical capture agents of interest include biotin, biotin derivatives, antigens such as fluorescein, digoxin, digoxigenin, and the like for which high affinity antibodies are available, and nucleic acid sequences. For the latter, unique capture sequences unrelated to the analyte sequence can be incorporated via a ligation reaction of a chimeric probe. Alternatively, extension reactions may also be employed, with the capture agent incorporated via a dNTP or a terminator or some other non-natural enzyme substrate.

Once a capture agent 142 has been incorporated, in a subsequent step 109, the modified probes are captured via the capture agent and sequestered, thereby allowing modified to be isolated from the unmodified probes. To perform the capture, a binding partner of the capture agent is provided on a solid support. The binding partner general forms a non-covalent complex with the capture agent. Representative examples of binding partners include avidin and streptavidin (for biotin derivatives), anti-fluorescein or anti-digoxigenin (or digoxin) antibodies or complementary polynucleotides. Such binding partners are provided attached to a solid support such as a bead, particle, container surface or gel and the like. After modified probes are bound to the binding partner, the unmodified probes may be washed away from the solid support, the solid support may be removed from the solution, or a flowing or moving current of the solution may pass by the support and the modified probes sequestered from the current by formation of a binding partner:capture agent complex. The result is to produce (111) a probe set 140 of modified probes 112b and 114b, that have been isolated from unmodified probes. The modified (and isolated) probes are detectable via the common sequence and signature sequence tags using any of the direct or indirect methods that are well known in the art.

Step 113 is similar to those of 101 and 107 except that the modification reaction is used to incorporate an enzyme resistant capping group 152. Again the reaction occurs at the reaction site 120 and requires the probe 112 or 114 to be hybridized to a target. Again, the reaction may be an extension reaction, a ligation reaction, or even a chemical ligation reaction, the result of which is to render the probe resistant to degradation by an enzyme, particularly an exonuclease. In this embodiment, the probes 112 and 114 preferably have capping group X₁ (128) present. Modification step 113 adds capping group X₂ (152) to the other end and thus with both ends ‘capped’, addition of an exonuclease to the sample solution will cause unmodified probes to be digested. The modified probes 112c and 114c will survive digestion to provide a set 150 of modified probes, again ready for detection via the common and signature sequence tags.

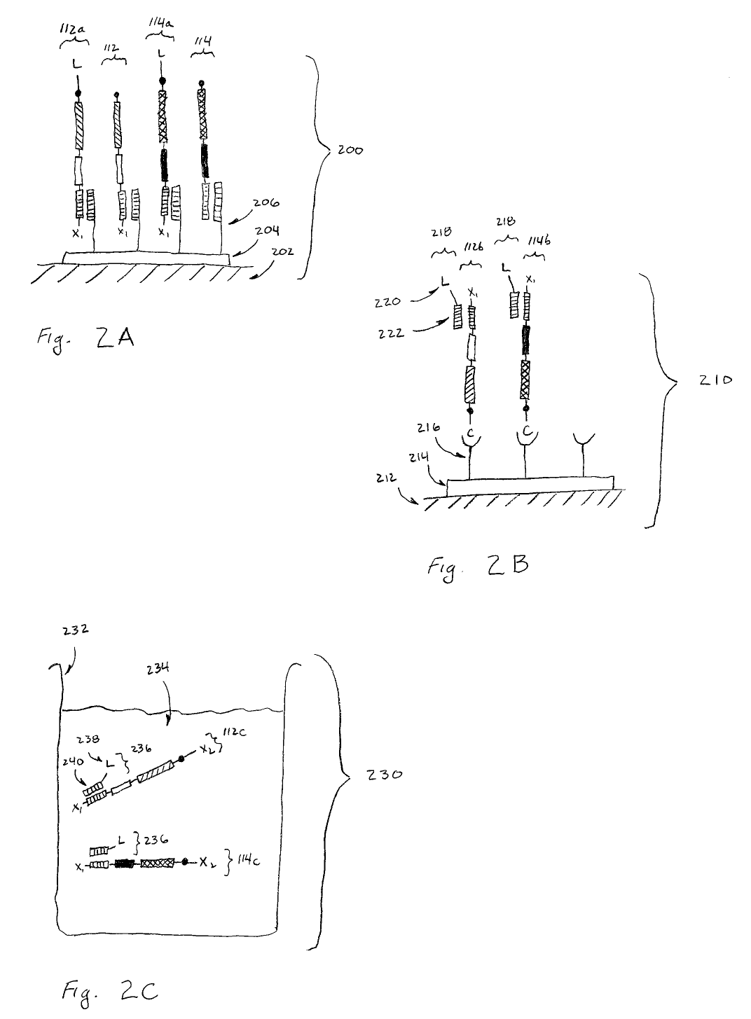

FIG. 2 illustrates exemplary methods for detecting the common sequence tag present in the modified probes to confirm the presence or absence of any of the plurality of polynucleotide analytes in the sample. In FIG. 2A, the modified set 130 is analyzed by an array hybridization process 200. FIG. 2A illustrates a planar array comprised of a support surface 202 and having patterned thereon a 1×1 array, with the array element 204 containing tag complement 206. As illustrated, the tag complement is comprised of a sequence substantially complementary, or more preferably, exactly complementary to the common sequence tag 126 of the probes 112a and 114a, and probes 112 and 114. In some instances the unmodified probes may still be present at this stage and may hybridize to the array, but even if so, they will not be detected because they lack the label agent. Probes 112a and 114a, which are labeled, will be detected either directly or indirectly, as per the well known methods of the art.

In FIG. 2B, the modified set 140 is analyzed by an array hybridization process 210. FIG. 2B illustrates a planar array comprised of a support surface 212 and having patterned thereon a 1×1 array, with the array element 214 containing binding partner 216. Note this binding partner may be the same partner discussed above in connection with FIG. 1, such that the binding and washing steps to isolate the probe set occurs on the array. This is particularly preferred if the strength of the binding between the capture agent and binding partner is strong, such as for biotin/streptavidin. Where the binding interaction is weaker or reversible, then the isolation may take place prior to analysis 210. Isolated probe set 140 is contacted with the array under binding conditions, and then interrogated with common sequence tag complementary probes 218. The complementary probes 218 are comprised of a sequence region 222 complementary to the common sequence tag and a label 220. The label 220 may be a primary label or a secondary label. Such methods are well known in the art of array hybridization detection, such as are discussed in U.S. Pat. No. 6,858,412, and is herein incorporated by reference.

In FIG. 2C, the modified set 150 is analyzed in a homogeneous solution process 230. The sample solution only contains the 112c and 114c probes after unmodified probes 112 and 114 were digested in step 115. To this solution is added common sequence tag complementary probes 236 that are comprised of a sequence region 240 complementary to the common sequence tag and a label 238. In this instance the label 238 must be capable of providing a signal that is modulated as a result of probe 236 hybridizing to probes 112c and 114c. Numerous methods are available, such as the hybridization protection assay and molecular beacons. Also, double strand specific antibodies can be used to detect hybridization of probe 236. In this instance, RNA/DNA heteroduplexes are preferred and one member of the hybridization pair must be an RNA polynucleotide. Also, other sequence detection methods may be favored in a homogeneous detection process, such as TaqMan, Invader, and cleavase reactions to produce mass tags or electrophoretic tags.

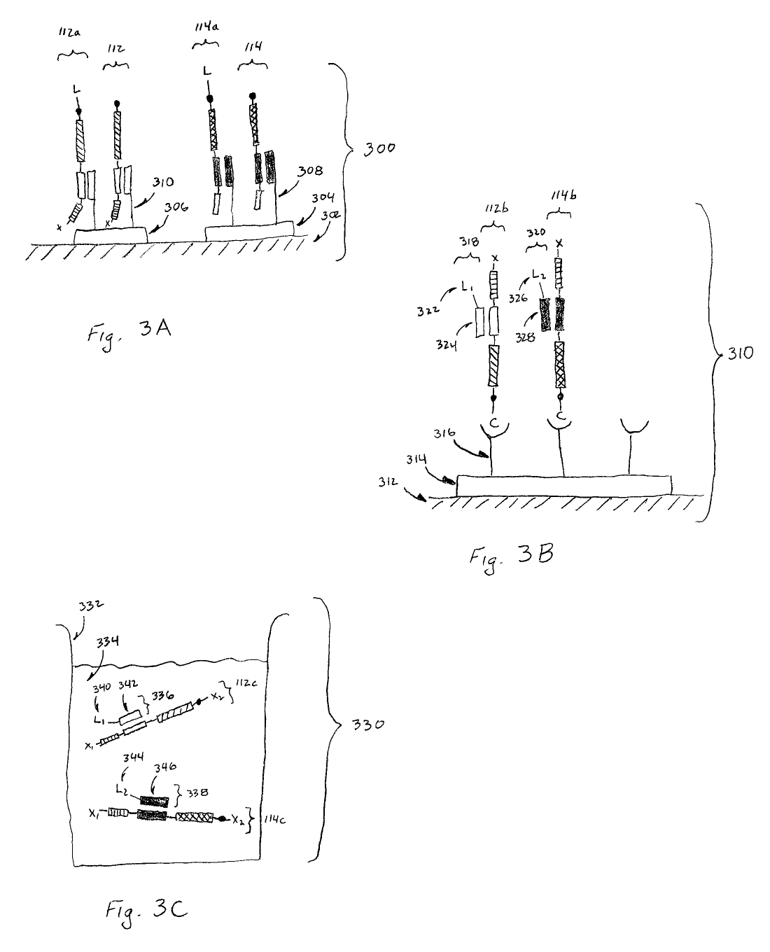

FIG. 3 illustrates exemplary methods for detecting the signature sequence tags present in the modified probes to confirm the presence or absence of each of the plurality of polynucleotide analytes in the sample. In FIG. 3A, the modified set 130 is analyzed by an array hybridization process 300. FIG. 3A illustrates a planar array comprised of a support surface 302 and having patterned thereon a 1×2 array, with array elements 304 and 306 containing tag complements 308 and 310, respectively. In this case the tag complements are substantially complementary, or more preferably, exactly complementary to the signature sequence tags 122 and 124 of the probes 112b and 114b. In this detection process the probes are segregated to addressable elements. Again, in some instances the unmodified probes may still be present at this stage, but even if so, they will not be detected because they lack the label agent. Probes 112a and 114a, which are labeled, will be detected either directly or indirectly, as per the well known methods of the art. Because each array element is addressable, the signal can be correlated to a specific probe and therefore to a particular analyte.

In FIG. 3B, the modified set 140 is analyzed by an array hybridization process 310. FIG. 3B illustrates a planar array comprised of a support surface 312 and having patterned thereon a 1×1 array, with the array element 314 containing binding partner 316. Note this may be the same array discussed above in connection with FIG. 2B. The same array used in detection process 210 may be reused in process 310. Here, the array with bound modified set 140 is interrogated with signature sequence tag complementary probes 318 and 320. These complementary probes 318 and 320 are comprised of sequence regions 324 and 328 complementary to the signature sequence tags 122 and 124 respectively, and labels 322 and 326. Labels 322 and 326 may be a primary label or a secondary label. Such methods are well known in the art of array hybridization detection, such as are discussed in U.S. Pat. No. 6,858,412, that is incorporated herein by reference.

In FIG. 3C, the modified set 150 is analyzed in a homogeneous solution process 330. To this solution is added signature sequence tag complementary probes 336 and 338. Again, these probes are comprised of sequence regions 342 and 346, and labels 340 and 344. Being a homogeneous solution process, labels 340 and 344 must be capable of providing a signal that is modulated as a result of probes 336 and 338 hybridizing to probes 112c and 114c. Numerous methods are available, as per the discussion in connection with FIG. 2C with the caveat that the method must be useful for multiplexed analysis. All the methods discussed are capable of providing 2 to 4 distinct signals for 2 to 4-plex analysis, and some provide highly multiplexed results by, for example, the detection of mass tags and electrophoretic tags. However, as will be understood from the discussion to follow, these methods do not require highly multiplexed detection methods in order to assay for the presence or absence of large numbers of analytes.

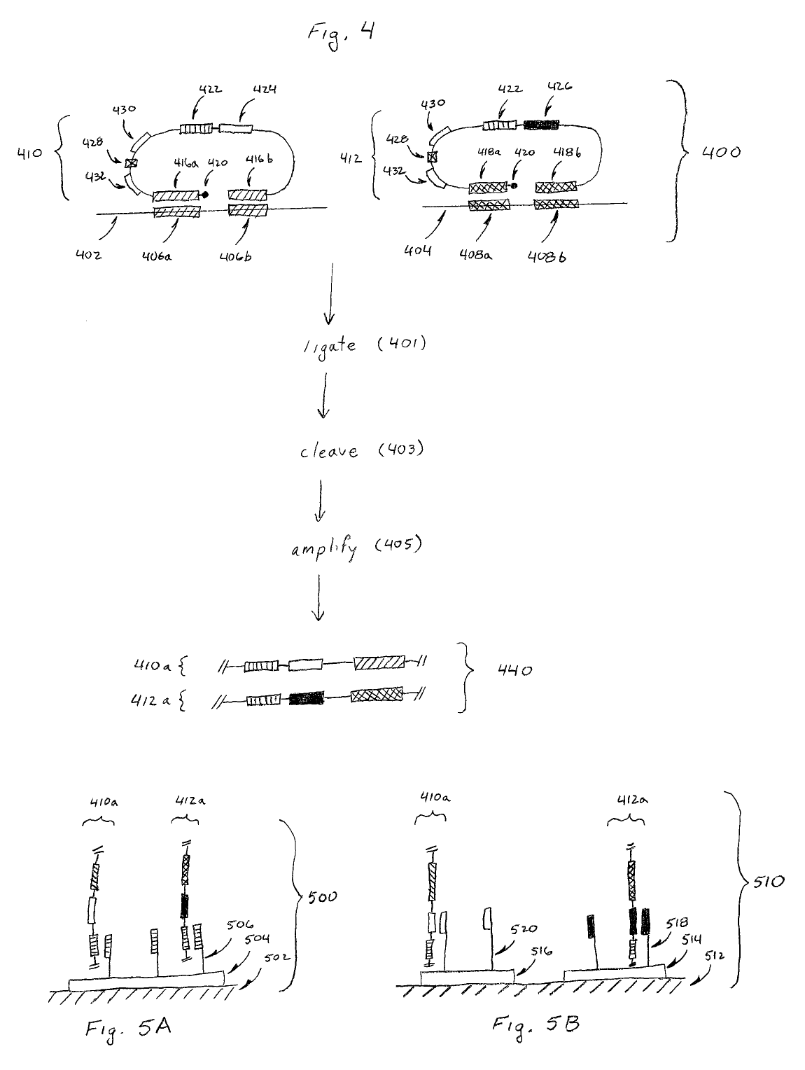

FIG. 4 illustrates a method by which, in another aspect of the invention, a probe set comprising probes 410 and 412 is mixed with sample suspected of containing target analytes 402 and 404, the probes are modified by cyclization as the result of a ligation reaction that occurs due to the presence of analyte(s), and any modified probes produced are subsequently amplified and made available for detection. An exemplary probe set with probes 410 and 412 is shown in the figure. Although the invention may be practiced with as few as two probes, typically at least 10 probes are used to assay for at least 10 analytes. More typically, the number of analytes assayed for is the range of 20 to 200, and the methods are useful for analyzing for at least 500 analytes, or as many as 5,000 to 100,000 and even between 100,000 and 1,000,000.

In this aspect of the invention employing cyclizable probes, each probe has two analyte-specific regions, 416a/416b and 418a/418b, by which the probe hybridizes to a particular analyte. Accordingly, each analyte contains two targeted sequence regions, 406a/406b and 408a/408b to which a probe will hybridize. The two regions in the analyte may be contiguous, in which case the two probe ends will abut each other, similar to a nicked site in a polynucleotide chain. Alternatively, the two regions may be separated by one or several nucleotide bases. One of the probe ends is identified as a reaction site. Chemical ligation reactions that can join together natural chain ends of polynucleotide structures are known in the art but in many cases activated functional groups are necessary precursors. Nonetheless, enzymatic reactions are the preferred method and therefore natural structures recognized by enzymes are preferred as the reaction site.

The probes are also comprised of a common sequence tag 422, signature sequence tags 424 and 426, optionally a cleavage site 428, at least one primer site 430 and optionally a second primer site 432. The common and signature sequence tags perform a similar role as discussed above. It should be noted that here, as well as for the methods illustrated with FIG. 1, more than one signature tag can be incorporated into a probe. Thus the two or more signature sequences embedded in a probe can be used to further categorize or specify the analyte being targeted by a particular probe. This concept is discussed more fully below in conjunction with sequence tag sets. Note also that despite the name, the common sequence tag may be represented by more than one sequence. Using a set of different, unique sequences as a common sequence tag can be useful to distinguish different classes or groups of probes. This also recognizes the fact that different probe sets may be combined for a single analysis. Other aspects of the probe features, structural characteristics and composition are also disclosed in U.S. Pat. No. 6,858,412.

The probe may also optionally comprise a first tag-adjacent sequence (usually restriction endonuclease sites and/or primer binding sites) for tailoring one end of the sequence tags (between 422 and 430), and second tag-adjacent sequence (between 424 and 416b, and 426 and 418b) for tailoring the other end of a labeled sequence tags. Alternatively, cleavage-site 428 may be added at a later step by amplification using a primer containing such a cleavage site.

In operation, solution 400 comprising probes 410 and 412 and targets 402 and 404 is mixed and the conditions and temperature adjusted as necessary to hybridize the probes with any target analyte present. Once hybrids have formed, in a next step 401 the probes are ligated to form cyclized probes. If there is a gap between the probe ends the gap must first be filled, generally by a polymerase extension reaction, a so-called “gap-filling reaction”, but a short oligonucleotide complementary to the target within the gap may also be used. The polymerase reaction is carried out by extending with a DNA polymerase a free 3′ end of one of the target-specific regions so that the extended end abuts the end of the other target-specific region, which has a 5′ phosphate, or like group, to permit ligation. In any case, after hybridization of the target-specific regions, and any gap-filling process, the ends of the abutting nucleotide regions are covalently linked by way of a ligation reaction using a ligase enzyme. The result is the creation of a cyclized, or circularized probe.

Following step 401, the now cyclized probe can serve as a template for rolling circle amplification. The resulting extended runoff product will contain tens or hundreds or more copies of the common and signature sequence regions as illustrated by modified probe set 440. Alternatively, the circularized probes can be cleaved at cleavage site 428 in step 403 to produce a linearized probe that now has its constituent features reordered. Cleavage site 428 and its corresponding cleaving agent is a design choice for one of ordinary skill in the art. In one aspect, cleavage site 428 is a segment containing a sequence of uracil-containing nucleotides and the cleavage agent is treatment with uracil-DNA glycosylase followed by heating. Such a reordered probe is also referred to as a “molecular inversion probe” due to the fact that what used to be the termini are now joined together and within the interior of the probe. This linearized probe is then amplified in step 405 by any of the amplification methods known in the art, particularly PCR, NASBA, TMA, CPT, SDA and the like to produce the set 440 of amplicons 410a and 412a comprised of multiple copies of the common and signature sequence tags. The modified (cyclized and linearized) probe can be amplified in the presence of unmodified probes because the unmodified probes cannot produce a competent amplicon. However the unmodified probes do serve as a template for an extension reaction or at least as a site for primer hybridization and thus may interfere with an efficient amplification reaction. Accordingly, the modified probes can be first isolated from unmodified probes following the ligation step 401 and before the cleavage step 403 by digesting remaining unmodified (linear) probes using an exonuclease. The modified (cyclized) probes are resistant to the actions of an exonuclease enzyme, and the resulting solution will be free of unmodified probes. Thus the subsequent amplification reaction will be maximized for efficient use of the reagents.

A multiplexed readout may be obtained from amplicons 440 by labeling and excising the common sequence tag 422 and/or the signature sequence tags 424 and 426, and specifically hybridizing the labeled tags to a microarray of tag complements, e.g. a GENFLEX array (Affymetrix, Santa Clara, Calif.); a bead array; or a fluid array, e.g. Chandler et al, U.S. Pat. No. 5,981,180 (Luminex, Austin, Tex.).

FIG. 5 illustrates array-based methods for detecting the tag sequences (common or signature) present in the modified (cyclized and amplified) probes such as those of set 440. Here the method is analogous to the methods previously described in conjunction FIGS. 2A and 3A. In FIG. 5A, the modified set 440 is analyzed by an array hybridization process 500. FIG. 5A illustrates a planar array comprised of a support surface 502 and having patterned thereon a 1×1 array, with the array element 504 containing tag complement 506, which is substantially complementary to the common sequence tag 422. The amplified probes 410a and 412a are contacted with the array under hybridization conditions, and the presence of hybridized probes is detected. Detection occurs via a label attached to or associated with the modified probes. The label may be either a direct or indirect label, according to methods well known in the art. A label may be incorporated during the amplification step, such as by use of a labeled probe or labeled dNTPs. The label may even be a polynucleotide for use as a hybridization region, which may be incorporated into amplicons by using a chimeric primer with the hybridization region in a 5′-tail. Alternatively, such a hybridization region may have designed into the original probes 410 and 412. A more detailed discussion of methods for preparing and detecting modified probes can be found in U.S. Pat. No. 6,858,412.

The process of FIG. 5A can be used to screen for the presence of any of the analytes of interest. If there is a positive identification, the array process 510 illustrated in FIG. 5B can be used to further identify which of the analytes is present. A support surface 512 has patterned thereon a 1×2 pattern, with elements 514 and 516 containing tag complements 518 and 520, respectively, that are substantially complementary to signature sequence tags 424 and 426, respectively. The labeling process enabling detection of a hybridization is the same as discussed for FIG. 5A. Indeed, the same solution can be used to contact with either type of array, an array with a common sequence tag complement or an array of signature sequence tag complements. The operations are identical, the difference is in the type of information obtained in the result.

A schematic illustration of a probe set containing sequence tag sets is shown in FIG. 6. Each probe in probe set 600 contains a sequence tag set 602, which, as illustrated, contains three tags 604, 606 and 608. The number of tags in a sequence tag set (STS) may be as few as one, and as many as are desired, although practical limits on the length of a probe will limit the number of tags in an STS. Typically an STS will have at least two but fewer than 5 tags.

Each tag in an STS may have any number of unique symbols at a tag position. The number of unique symbols used in a numeric system is referred to in mathematics as the radix. For example, the decimal system has radix 10 in that 10 unique symbols (0 through 9) are used in each position. Other radix systems are common, such as binary (2 symbols) and hexadecimal (16 symbols) and illustrate the idea that any number of unique symbols can be used to represent values. In the case of STSs, the unique symbol is a nucleotide sequence of, for example, 5 to 40 bases. As is well known in the art, an extremely large number of unique nucleotide sequences can be generated for such lengths. For example, there are 220 different sequences for a ten base sequence. Although not all unique sequences are equally useful because of secondary structure, the possibility of cross-hybridization with other probes or self-hybridization or hairpinning, or because the sequence might be homologous to analyte sequences, the number of useful, unique sequences is smaller but still sufficiently large for the purpose of constructing STSs.

Sets of arbitrary sequences have been developed for use with addressable arrays, some of which are referred to as “zipcodes” or “barcode sequences”, are well known in the art and can serve as the unique sequence symbols as a tag of an STS. Other arbitrary sequences can be readily constructed, using algorithms known in the art to select for probe sets that have minimal cross-interactions, self-interactions and do not promote non-specific interactions with analyte sequences because of partial complementarity.

STSs may have the same number of unique sequences (symbols) for each tag, or the number of unique sequences may vary for each tag. As shown in FIG. 6, the first tag 604 has only one unique sequence, symbolized as “A” (622), whereas the second and third tags 606 and 608 each have three unique sequences, respectively symbolized as “1” (624), “2” and “3”, and “a” (626), “b” and “c”. One difference to note between STSs and typical numerical systems is that each symbol of each tag of an STS is unique whereas the same symbols are typically used for each digit in a numerical system.

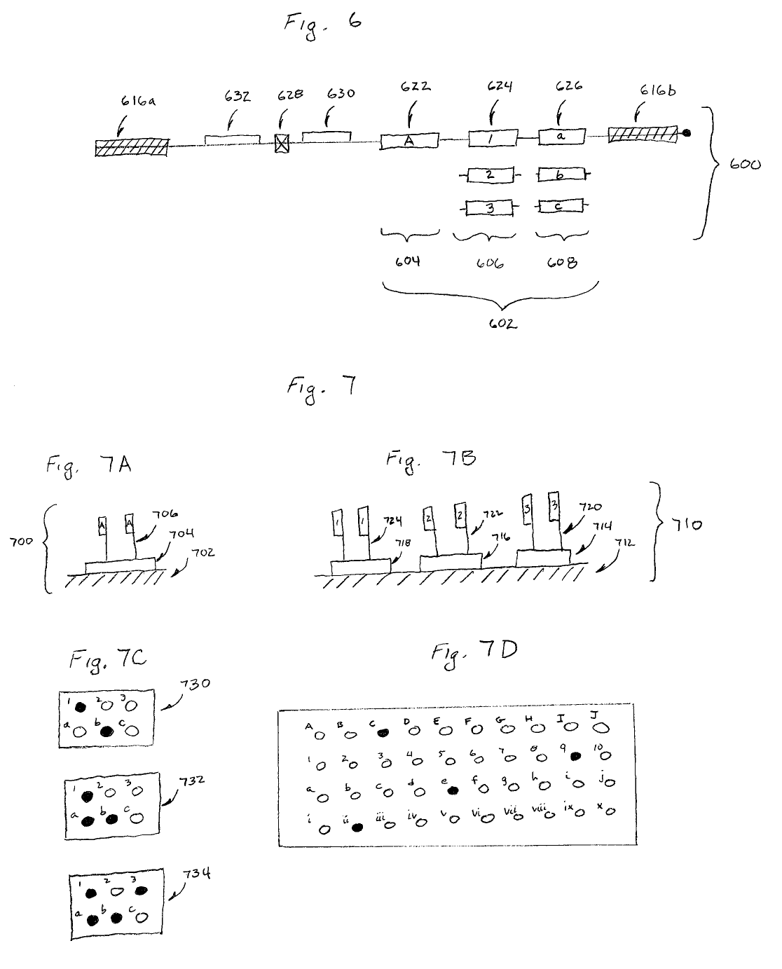

The STS of FIG. 6 allows for up to nine different unique tags. In this case, the first tag 604, with only one symbol used for the tag, functions as a common sequence tag as described above. In an assay in which all nine possible tags are used in probes for nine different analytes, detecting the sequence of “A” (622) will provide information as to whether any, or none, of the nine analytes are present in the sample. If a first stage detection of “A” reveals one of the analytes is present, then the same processed sample can be used in a second stage detection step to further assay for the identities of either subsets of analytes or individual analytes. Methods and arrays useful for the analysis of STSs are further described in FIG. 7.

The remainder of each probe of probe set 600 as illustrated also comprise analyte-specific regions 616a and 616b, primer sites 630 and 632, and cleavage site 628. The relative location of these enumerated features is the same as described earlier in conjunction with FIG. 4. Furthermore, these features can be present and used, or present or absent and not used in performing the method, all in accordance with the description given earlier. Note that probes of the type shown in FIG. 1, which are effective with one analyte-specific region, can also comprise STSs as described here. In fact, the common sequence tags and signature sequence tags described in FIG. 1 are a special case of STS having two digits, the first of which is represented by one symbol.

The relative ordering of the tags 604, 606 and 608 of STS 602 does not matter. The tags may be contiguous sequences, or they may be separated by 1, 5, 10 or more bases. Whether the tags are contiguous or not, an alternative detection scheme contemplates the detection of adjacent sequence portions of two digits. For example, an array element would have a tag complement that is complementary to e.g. the 3′ half of a first tag and the 5′ half of the following tag, thus affording the detection of the combination of a particular first and second tag combination. The probe sense can obviously be reversed, and the complement of any intervening sequence between the tags can be incorporated in the tag complement on the array. If the probe is to be amplified in performing the method, then the tags should be located within the portion of the probe that will be amplified. In the probes illustrated in FIG. 6, the tags 604, 606 and 608 should be located between analyte-specific region 616a and primer region 632, and/or primer region 630 and analyte-specific region 616b. In some instances, a primer sequence region may also function as the tag of an STS. In many instances, such a tag could be common to the entire probe set.

FIG. 7 illustrates exemplary arrays useful for the detection of tag sequences present in modified probes (FIGS. 7A, 7B) and representations of exemplary results obtained from an array (FIGS. 7C, D). The arrays used to perform the analysis may be either planar arrays or random arrays, as referred to earlier. FIG. 7A illustrates a planar array 700 comprised of a support surface 702 and having patterned thereon a 1×1 array, with the array element 704 containing tag complement 706. As illustrated, the tag complement is comprised of a sequence “A′” that is complementary to the sequence “A” (622) of tag 604 of the probe 600. Thus array 700 functions to screen for the presence or absence of any of the analytes targeted by probe set 600.

In FIG. 7B, a planar array 710 comprised of a support surface 712 has patterned thereon a 1×3 array, with array elements 714, 716 and 718 containing, respectively, tag complements 720, 722 and 724 having sequences “1′”, “2′” and “3′”. The tag complements are used to detect the set of sequences found in second tag 606. Thus, for example, if analytes targeted by probes with an STS of “A1a”, “A1b” or “A1c” were present, then the first array spot with tag complement “1′” (724) would be positive.

Array 710 could be used in a first detection step to stratify samples according to which subsets of the analytes were present, e.g. the subset with STS tag “1”, “2” or “3”. It should thus be appreciated that the STSs should be assigned to targets according to a particular scheme such that the detection and stratification of subsets or subgroups provide useful, actionable information. The number of nested subsets and subgroups can be scaled as necessary, depending on the type of information sought within the pool of analytes. Array 710 can also be used in a second detection step, following an initial screen by e.g. array 700, in which the presence of some member of the probe set has already been determined. Generally, arrays can be prepared to detect the members of any one tag in an STS, or any two or more tags, or of the entire set of tags of an STS. The determination of which subset of the tags to interrogate in any one step is entirely determined by the needs of the operator. Furthermore, should it be necessary to more specifically determine the identity of an analyte, then the same processed sample can be used in a subsequent determination using a different array to interrogate a different group of tags.

FIG. 7C illustrates a representation of data obtained from an analysis of two tags of an STS. For the sake of convenience, the probe set 600 is employed, and tags 606 and 608 which each have three members. The figure represent a series of 2×3 arrays, 730, 732 and 734, in which the row of each array interrogates the members of a tag. The spots are labeled “1”, “2” and “3”, and “a”, “b” and “c”, with a dark spot indicating a positive result and a open circle indicating a negative result. In array 730 the result is unambiguous because only one spot in each row is positive. Thus, the assay reveals that the analyte targeted by the probe with an STS of “A1b” is the only analyte present. In array 732, the result is still unambiguous, and the pattern reveals that two analytes, corresponding to “A1a” and “A1b”, are both present. In array 734, the pattern does not specifically determine which analytes are present. However, the array does reveal that analytes from subgroup “2” are not present, nor are those from subgroup “c”. A read of the pattern indicates that up to four analytes (“A1a”, “A1b”, “A3a”, A3b”) might be present but at least two are present (“A1a” or “A1b”, and “A3a” or A3b”). If more specific identification is required then tag complements spanning adjacent tags can be used, or the probes can be fractionated (e.g. by affinity methods) and re-analyzed, or analyzed by other methods such as mass spec, electrophoresis and the like.

FIG. 7D illustrates a larger, scaled up version of this type of analysis with STSs. Represented is a 4×10 array, which could be used to screen the results from a probe set with an STS of four tags, with 10 unique symbols each, or a probe with an STS of ten digits, with 4 unique symbols each. The former is a more preferable manner of encoding 10,000 probes because the probe with four digits will be shorter and thus less difficult and costly to prepare. However, other considerations, such the mapping of the information content to be assayed for into STSs may dictate more tags rather than fewer. The pattern illustrated reveals that the analyte corresponding to “C9e-ii” is present. This illustrates the power of the method in analyzing for the presence of one or a few analytes within an enormous set of possibilities, here, 10,000 possible analytes. Thus, 10,000 probes are used to assay for 10,000 analytes, but the detection step is very efficient: the results are obtained from a 10×4 array (40-element array), rather than a 10⁴-element array. Furthermore, by stratifying and categorizing the analytes through the encoding of the STSs, a useful answer may arise from simply interrogating one tag. For example, if the required information was which group of thousand is any analyte representative of, then a 1×10 array would be sufficient to determine which particular group of thousand any analyte present was a member of.

Thus, another aspect of invention is a method of detecting the presence or absence of members of sets of polynucleotide analytes in a sample, comprising: (a) mixing the sample with a set of polynucleotide probes, each comprised of a sequence at which the probe can hybridize with one of the plurality of polynucleotide analytes, a unique sequence tag set comprising at least two tags, and a reaction site at which the probe can be modified only when the probe is hybridized to a polynucleotide analyte; (b) after allowing the probes to hybridize to any polynucleotide analyte that may be present, modifying any probes so hybridized; and (c) analyzing for the presence of the members of at least one of the tags in the modified probes to determine if any of the members of a set of polynucleotide analytes are present.

In another aspect, the above method of detecting the presence or absence of members of sets of polynucleotide analytes in a sample further comprises: in said analyzing step, contacting said modified probes with an array comprising one spot for each of said tags represented in said sequence tag sets of said set of probes; and reading the pattern of hybridization on the array to determine which of said polynucleotide analytes are present, which might be present and which are absent.

The above teachings are intended to illustrate the invention and do not by their details limit the scope of the claims of the invention. While preferred illustrative embodiments of the present invention are described, it will be apparent to one skilled in the art that various changes and modifications may be made therein without departing from the invention, and it is intended in the appended claims to cover all such changes and modifications that fall within the true spirit and scope of the invention.

What is claimed is:

1. A method of detecting the presence or absence of any of a plurality of polynucleotide analytes in a sample, comprising:
(a) mixing the sample with a probe set, comprised of at least two polynucleotide probes, wherein each polynucleotide probe comprises of a sequence that is complementary to one of the plurality of polynucleotide analytes, a common sequence tag that is common to all probes in the probe set, and a reaction site at which the probe can be modified only when the probe is hybridized to a polynucleotide analyte;
(b) allowing the probes to hybridize to polynucleotide analytes in the sample;
(c) modifying any probes so hybridized; and
(d) determining if the common sequence tag is present in the modified probes, wherein the presence of the common sequence tag in the modified probes is indicative of the presence of at least one of the plurality of polynucleotide analytes in the sample.

2. The method of claim 1, wherein said plurality of analytes is greater than 10.

3. The method of claim 1, wherein said plurality of analytes is in the range of 20 to 200.

4. The method of claim 1, wherein said plurality of analytes is greater than 500.

5. The method of claim 1, wherein said plurality of analytes is in the range of 5,000 to 100,000.

6. The method of claim 1, wherein said modifying is performed by at least one enzymatic reaction.

7. The method of claim 6, wherein said at least one enzymatic reaction is a ligation reaction.

8. The method of claim 7, wherein said modified probes are circular probes.

9. The method of claim 1, wherein said detecting step comprises isolating modified probes from unmodified probes.

10. The method of claim 9, wherein said isolating occurs by enzymatic degradation of said unmodified probes.

11. The method of claim 10, wherein said detecting step further comprises amplifying the common sequence tag by PCR.

12. The method of claim 1, wherein each probe is a molecular inversion probe comprising:
a) a first targeting sequence;
b) a second targeting sequence; and
c) a cleavage site;
wherein said first and second targeting sequences hybridize to one of said plurality of polynucleotide analytes.

13. The method of claim 12 wherein said modified probes are circularized molecular inversion probes.

14. A method of detecting the presence or absence of each of a plurality of polynucleotide analytes in a sample, comprising:
(a) mixing the sample with a set of polynucleotide probes, each comprised of a sequence at which the probe can hybridize with one of the plurality of polynucleotide analytes, a common sequence tag, a signature sequence tag, and a reaction site at which the probe can be modified only when the probe is hybridized to a polynucleotide analyte;
(b) after allowing the probes to hybridize to any polynucleotide analyte that may be present, modifying any probes so hybridized;
(c) detecting the common sequence tags present in the modified probes to determine if any of the polynucleotide analytes are present; and
(d) whenever any of the plurality of polynucleotide analytes is determined to be present, detecting each of the signature sequence tags present in the modified probes, whereby the presence of each of the plurality of polynucleotide analytes is determined.

15. The method of claim 14, wherein said modifying comprises a ligation reaction.

16. The method of claim 14, wherein said modified probe is a circular probe.

17. The method of claim 14, wherein each polynucleotide probe is a molecular inversion probe comprising:
a) a first targeting sequence;
b) a second targeting sequence; and
c) a cleavage site;
wherein said first and second targeting sequences hybridize to one of said plurality of polynucleotide analytes.

18. The method of claim 14, wherein said detecting of signature sequences in said modified probes comprises amplifying said signature sequences, contacting the amplified products with a capture probe array, and detecting hybrids formed on the array between the amplified products and the capture probes.

19. A method of detecting if any of a plurality of polynucleotide targets is present in a sample, said method comprising:
a) mixing the sample with a set of probes under reaction conditions so that probes in the set that are specifically hybridized to a target in the sample, are modified to form selectable probes, each selectable probe comprising a first common priming site, a second common priming site and a common sequence tag between said first and second common priming sites;
b) isolating the selectable probes from the probes that are not modified;
c) amplifying the selectable probes isolated in b); and
d) determining if any of the amplified selectable probes are present, whereby the presence of any of the plurality of polynucleotide targets is determined by the presence of an amplified selectable probe.

20. The method of claim 19 further comprising determining if a specific polynucleotide target is present in the sample and wherein said selectable probes further comprise a signature sequence region between said first and second common priming sites, by:
a) contacting under hybridization conditions said amplified selectable probes with a solid support containing an array of end-attached capture probes each comprising a capture region substantially complementary to one of the signature sequence regions; and
b) detecting hybrids formed on the array;
whereby the presence of a specific polynucleotide target in said sample is determined by the formation of a hybrid between a selectable probe and a complementary capture probe on the array.
